(19) United States
(12) Patent Application Publication
(10) Pub. No.: US 2009/0216092 A1
(43) Pub. Date: Aug. 27, 2009

(54) SYSTEM FOR ANALYZING EYE RESPONSES TO ACCURATELY DETECT DECEPTION

(75) Inventors: Ronald Waldorf, Beverly Hills, CA (US); Barbara Mauch, Inglewood, CA (US)

Correspondence Address: PATTON BOGGS LLP, 1801 CALFORNIA STREET, SUITE 4900, DENVER, CO, 80202, US

(73) Assignee: AcuNetx, Inc., Torrance, CA (US)

(21) Appl. No.: 12/037,686

(22) Filed: Feb. 26, 2008

(51) Int. Cl. A61B 3/11; A61B 5/02

(52) U.S. Cl. 600/301; 600/558

(57) ABSTRACT

The present system for analyzing eye responses to automatically detect deception obtains a series of digital image frames from a headset. This system precisely controls the illumination generated by the headset to bias the subject's pupils and automatically and precisely determine the subject's pupil location, size, and responsiveness. These measurements enable the system for analyzing eye responses to automatically detect deception by accurately measuring the subject's involuntary pupillary responses to questions posed by an examiner. These measurements can be used exclusively or in combination with one or more of the standard elements of the polygraph machine to automatically detect deception.

### CROSS-REFERENCE TO RELATED APPLICATIONS

This application is related to U.S. patent application Ser. No. 11/934,414 filed Nov. 2, 2007 and titled “System For Analyzing Eye Responses To Automatically Track Size, Location And Movement Of The Pupil”; U.S. patent application Ser. No. 11/934,539 filed Nov. 2, 2007 and titled “System For Analyzing Eye Responses To Automatically Determine Impairment Of A Subject”; and U.S. patent application Ser. No. 11/398,009 filed Apr. 5, 2006 and titled “Image-Based System To Observe And Document Eye Responses”.

### FIELD OF THE INVENTION

This invention relates to a system for non-invasively tracking the size, location, and movement of a subject's pupil to implement a valid noninvasive psychophysiological test to accurately detect deception.

### BACKGROUND OF THE INVENTION

It is a problem to accurately determine whether a subject is providing deceptive answers to questions posed by an interviewer (also termed “examiner” herein). One commonly used instrument to assist in making this determination is a polygraph, which is basically a combination of medical devices that are used to monitor physiological changes occurring in the subject's body. As a subject is questioned about a certain event or incident, the examiner monitors the change in the subject's heart rate, blood pressure, respiratory rate and electro-dermal activity (sweatiness in the fingers) compared to the subject's normal levels. Fluctuations in these various monitored physiological characteristics may indicate that the subject is being deceptive, but the exam results must be interpreted by the examiner. Polygraph exams are most often associated with criminal investigations, but they are also used for employment screening.

Polygraph examinations are designed to look for significant physiological responses in a subject's body in response to stress, such as the stress associated with deception. The polygraph exams are not able to specifically detect if a person is lying, but there are certain physiological responses that most persons undergo when attempting to deceive another person. By asking questions about a particular issue under investigation and examining a subject's physiological reactions to those questions, a polygraph examiner can determine if deceptive behavior is being demonstrated by the subject.

In administering a polygraph exam, several tubes and wires are connected to the subject's body in specific locations to monitor a set of the subject's physiological characteristics. Deceptive behavior is supposed to trigger certain physiological changes in the subject that can be detected by a polygraph and a trained polygraph examiner, who is sometimes called a forensic psychophysiologist (FP). This polygraph examiner is looking for the amount of fluctuation in certain physiological activities:
Respiratory rate—Two pneumographs, rubber tubes filled with air, are placed around the subject's chest and abdomen. When the chest or abdominal muscles expand, the air inside the tubes is displaced and transducers convert the energy of the displaced air into electronic signals.
Blood pressure/heart rate—A blood-pressure cuff is placed around the subject's upper arm. Tubing runs from the cuff to the polygraph. As blood pumps through the subject's arm it makes sound; the changes in pressure caused by the sound displace the air in the tubes, which are connected to transducers which convert the pressure changes into electrical signals.
Galvanic skin resistance (GSR)—This is also called electro-dermal activity, and is basically a measure of the perspiration on the subject's fingertips. The finger tips are one of the most porous areas on the body and are a good place to look for perspiration. Fingerplates galvanometers) are attached to two of the subject's fingers. These plates measure the skin's ability to conduct electricity. When the skin is hydrated (as with perspiration), it conducts electricity much more easily than when it is dry.

Some polygraphs also record the subject's arm and leg movements. As the polygraph examiner asks questions, signals from the sensors connected to the subject's body are recorded on a single strip of moving paper or in a digital memory. However, the polygraph does not measure truth-telling; it measures changes in the subject's blood pressure, breath rate and perspiration rate, but those physiological changes can be triggered by a wide range of emotions. The polygraph machine measures various physiological changes, including in blood pressure and heart rate, to determine when the subject is getting anxious, based on the idea that deception involves an element of anxiety. Since an emotion, such as anxiety, can be triggered by many factors other than lying, the polygraph tests can overlook smooth-talking liars while pointing a finger at innocent people who just happen to be rattled by the test. In addition, there are subjects who can control their blood pressure, breath rate and perspiration rate and thereby thwart the effectiveness of the polygraph test, since these physiological characteristics are not involuntary responses.

Therefore, there is no viable automated system that can accurately measure various physiological changes in a subject in response to questions posed by an examiner, where the physiological changes in the subject are involuntary in nature and therefore not susceptible to the subjects control.

### BRIEF SUMMARY OF THE INVENTION

The above-described problems are solved and a technical advance achieved by the present system for analyzing eye responses to accurately detect deception (termed “Deception Detection System” herein). The measurement of a subject's eye responses has use in various applications, and one of particular interest is to accurately determine deception by the subject in responding to questions posed by an examiner. These measured eye responses are involuntary physiological responses of the subject to stress, such as the stress associated with deception, and include pupillary responses for either eye or both eyes simultaneously. These measurements can be used exclusively or in combination with one or more of the standard elements of the polygraph machine to accurately detect deception.

The Deception Detection System obtains a series of digital image frames from the subject, using a headset, which series of digital image frames capture pupil location, size and responsiveness in response to a set of questions posed to the subject by an examiner. A typical headset that can be used for this purpose is illustrated in U.S. patent application Ser. No. 11/398,009 and it generates the series of digital image frames for use in detecting deception. This headset has light emitting diodes (LEDs) to act as illumination sources and image generation devices (cameras) to capture the subject's pupillary responses. The resulting image frames are digitized for analysis by a Pupil Detection and Response Process (105 in FIG. 1), such as that described in U.S. patent application Ser. No. 11/934,414, where the size and reactivity of the pupil are the primary data that is retrieved from the sequence of digital images. Thus, the Pupil Detection and Response Process can determine the subject's pupil area and responsiveness, which data can be used, for example, to determine a subject's attempts at deception.

This Pupil Detection and Response Process thereby produces pupil area and location data which can be used by the Deception Detection System to determine the subject's attempts at deception by automatically monitoring the subject's ocular responses when the examiner poses a series of questions to the subject. An important aspect of the Deception Detection System is the Pupil Bias System which provides the ability to bias the subject's pupil size by providing a controllable level of illumination to ensure that the subject's pupils are not overly or under dilated, which would limit the ability of the Pupil Detection and Response Process to detect the greatest range of pupil reaction. All other known pupil monitoring systems either do not care about controlling ambient light impinging on the pupil or test the subject in a totally dark pupil environment. The present Deception Detection System provides a small amount of light that slightly constricts the pupil but does not compromise its ability to be physiologically dilated. This Pupil Bias System is unknown in the existing art of pupillomtery. The Pupil Bias System can either use a self-contained headset (as shown in the above-noted U.S. patent application Ser. No. 11/398,009) which eliminates ambient light and provides the pupil biasing illumination all within the same ‘chamber’. Alternatively, the Pupil Bias System can control the ambient light in the room where the subject is being questioned to a low-light level that would cause the pupil to slightly constrict, but not enough to preclude its dilation.

Thus, the combination of precisely controlling the illumination to bias the subject's pupils and the ability to automatically and precisely determine the subject's pupil size, and responsiveness, provides the Deception Detection System with the ability to accurately measure the subject's involuntary pupillary responses to questions posed by an examiner and thereby detect attempts at deception by the subject.

The Deception Detection System can optionally include or be connected to a standard polygraph machine, in order to provide the examiner with a plurality of streams of data from which the subject's deception can be determined. The Deception Detection System generates output data which is indicative of the subject's pupillary response, which the examiner can correlate with the Respiratory Rate, Blood Pressure/Heart Rate, and Galvanic Skin Resistance measurements. In this manner multiple streams of data can be correlated to enhance the accuracy of the ultimate determination.

### BRIEF DESCRIPTION OF THE DRAWINGS

FIG. 1 illustrates a block diagram of the present Deception Detection System, including the Pupil Detection and Response Process and the Pupil Bias System;

FIG. 2 illustrates, in flow diagram form, the operation of the present Deception Detection System which uses the measured pupil responsiveness data generated by the pupil tracking system to generate an indication of deception; and

FIGS. 3 and 4 illustrate side and front views, respectively, of a typical headset used in the present Deception Detection System.

### DETAILED DESCRIPTION OF THE INVENTION

It is a problem to accurately determine whether a subject is providing deceptive answers to questions posed by an interviewer (also termed “examiner” herein). One commonly used instrument to assist in making this determination is a polygraph, which is basically a combination of medical devices that are used to monitor physiological changes occurring in the subject's body. As a subject is questioned about a certain event or incident, the examiner monitors the change in the subject's heart rate, blood pressure, respiratory rate and electro-dermal activity (sweatiness in the fingers) compared to the subject's normal levels. Fluctuations in these various monitored physiological characteristics may indicate that person is being deceptive, but the exam results must be interpreted by the examiner, which is a potential source of error. Additionally, the polygraph machine measures various physiological changes, including in blood pressure and heart rate, to determine when the subject is getting anxious, based on the idea that deception involves an element of anxiety. Since an emotion, such as anxiety, can be triggered by many factors other than lying, the polygraph tests can overlook smooth-talking liars while pointing a finger at innocent people who just happen to be rattled by the test. In addition, there are subjects who can control their blood pressure, breath rate and perspiration rate and thereby thwart the effectiveness of the polygraph test, since these physiological characteristics are not involuntary responses.

It is proposed that the determination of a subject's attempts at deception can be detected by the measurement of the subject's eye behavior, which measurements are compared to a normal response for the subject since pupil dilation and pupil movement are involuntary responses. However, the accurate measurement and tracking of the subject's pupil centroid location and determination of the area of the pupil are critical to the precise determination of deception. It is also important to detect eye blinking in order to ensure that pupil measurements are not computed at those time instances when a blink occurs, since the pupil is either partially visible or not visible at all when a blink occurs.

### Headset

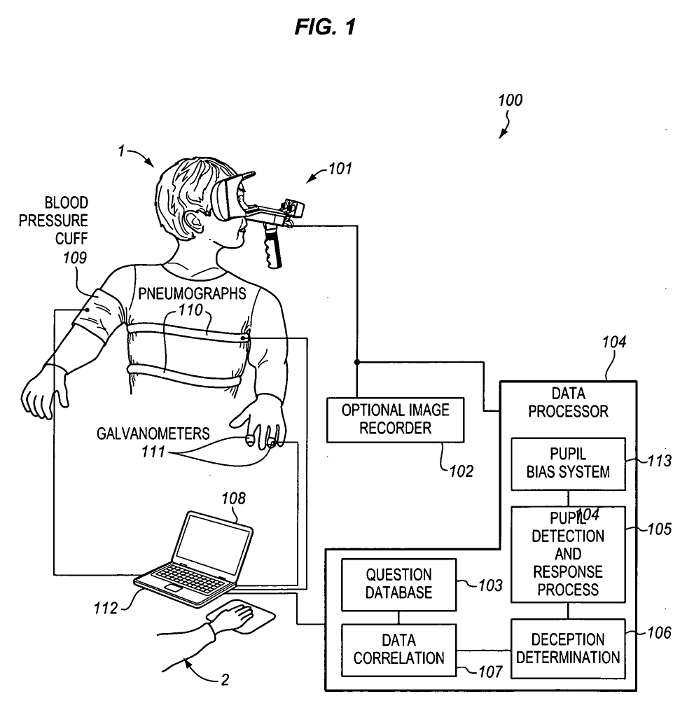

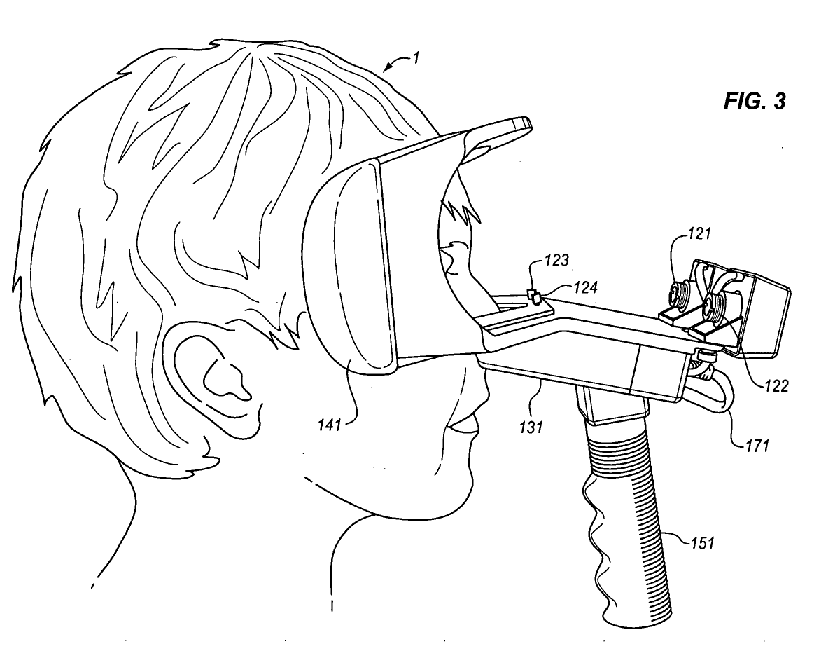

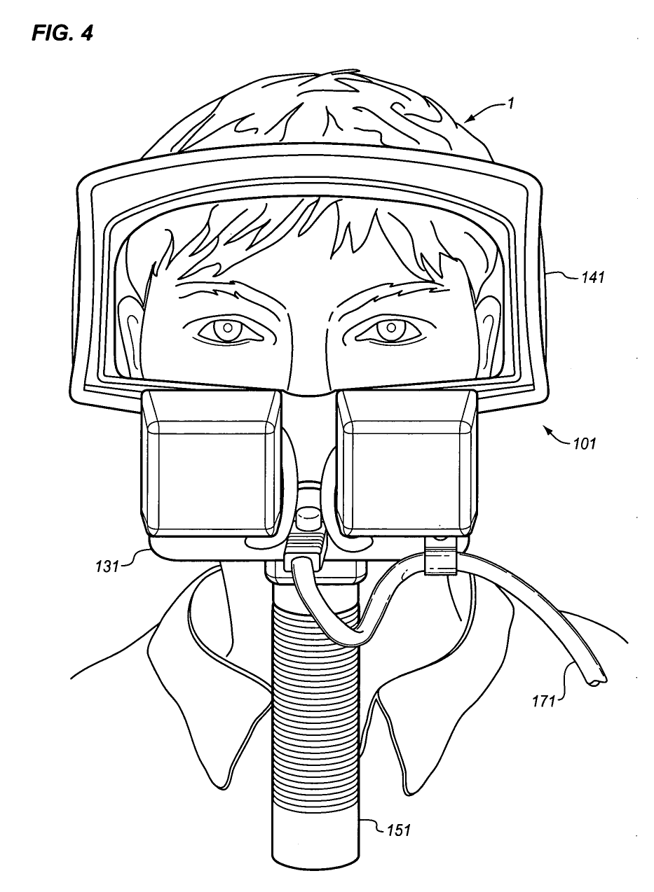

FIG. 1 illustrates a block diagram of the Deception Detection System 100, where a headset 101 is used to generate a series of digital image frames to capture a subject's pupil location and size. The headset 101 (as shown in FIGS. 3 and 4) includes illumination devices 123-124 and imaging devices 121, 122 that are attached to a frame 131, which includes a faceplate 141 to position the subject's face with respect to illumination devices 123-124 and imaging devices 121, 122. A source of electrical power, such as batteries, is provided to power illumination devices 123-124 and imaging devices 121, 122, with the batteries typically located in the grip portion 151 of the headset 101. To provide proper illumination bias of the subject's eyes, control signals are transmitted to the headset 101 by the Pupil Bias System 113 of the data processor 104 to manage the light output of illumination devices 123-124 to provide approximately 1 Ft-Candle of diffuse, illumination. The imaging devices 121, 122 can optionally be connected to an image recording device 102 for capturing and storing image data if a record of the images is desired. The interconnection between the headset 101 and the image recording device 102 can be wired or wireless, using a cable or wireless communication protocol, respectively.

The headset 101 is based on a frame 131 to which the various elements are attached. A faceplate 141 is attached to the frame 131, shaped to substantially conform to a subject's face and having two eye openings 142, 143 oriented opposite a subject's eyes when the subject places their face against faceplate 141. A chin rest 144 is also provided to assist in the alignment of the subject's face with the eye openings 142, 143. Attached to frame 131 are two imaging devices 121, 122, such as image cameras of the Charge Coupled Device type (or other functional equivalent), one that provides an image of the right eye and the other of the left eye. The resultant image provides data for the proper determination of eye gaze tracking and the monitoring of eye behavior. The orientation of the imaging devices 121, 122 can be adjusted to place the eyes in the center of the image that is displayed on the image display device 108. The image display device 108 can be of the quad-type, meaning that each eye is displayed in one of four image quadrants on the screen of the image display device 108—typically next to each other on the upper horizontal or lower horizontal aspect ratio to give an image as if the eyes were seen as normal, adjacent to each other.

The faceplate 131 contains illumination devices 123-124, such as infrared Light Emitting Diodes, for the illumination of the subject's eyes. Two light emitting diodes can be used in or adjacent to each eye opening 132, 133 to ensure proper illumination of the subject's eyes. These infrared Light Emitting Diodes produce invisible illumination. One unique attribute of the infrared illumination is the visual observational enhancement of the CCD image of the eye for dark-iris individuals.

### Polygraph

The Deception Detection System 100 can optionally include a polygraph 112 in whole or part. As shown in FIG. 1, the subject 1 is equipped with two pneumographs 110, rubber tubes filled with air, placed around the subject's chest and abdomen. In addition, a blood-pressure cuff 109 is placed around the subject's upper arm. Tubing runs from the blood-pressure cuff 109 to the polygraph machine 112. Finally, fingerplates galvanometers) 111 are attached to two of the subject's fingers. These are the standard polygraph sensor elements and some or all of these may not be used in the Deception Detection System 100 to supplement the monitoring of pupillary responses of the subject. The polygraph machine 112 includes software to process the data generated by the above-noted sensor elements and the polygraph machine 112 includes a display 108, which can be shared with the other elements of the Deception Detection System 100 in order to display the generated data to the examiner 2. Thus, the examiner is provided with a real-time display of the subject's response to questions posed by the examiner as well as the subject's response during the period where the subject answers the question. Where the polygraph data is displayed along with the pupillary data, the examiner is provided with a multiplicity of sources of information from which to make a decision about the deceptiveness of the subject. As described herein, the Deception Detection System 100 can include a deception determination process 106 which processes the pupillary data and compares it with various metrics to make an automatic determination of deception, without the necessity of having input from the examiner.

### Pupil Detection and Response Process

Data processing system 104 processes the images received from the headset 101 using the Pupil Detection and Response Process 105. The Pupil Detection and Response Process 105 performs an analysis of the digital image frames generated by the imaging devices as they are received to quickly and accurately localize the pupil's boundaries, area, and center coordinates in close-up digital imagery of the subject's eye(s) as captured with the imaging devices. The Pupil Detection and Response Process 105 can be implemented using, in whole or in part, the process disclosed in U.S. patent application Ser. No. 11/934,414, which is described herein for the purpose of illustrating the operation of this process.

The Pupil Detection and Response Process 105 uses a method for quickly and accurately localizing the subject's pupils, as well as their boundaries, area, and center coordinates. The Deception Detection System 100 receives a series of digital image frames of the subject's eyes and the Pupil Detection and Response Process 105 uses a region-growing, eye-response tracking process to extract and track the centroid location and area of the subject's pupil(s) under difficult conditions, such as blinking, eyelid droop, head movement, eye gaze at the extreme corners of the eyes, presence of heavy eye makeup, and where light specularities occur in or near the pupil region.

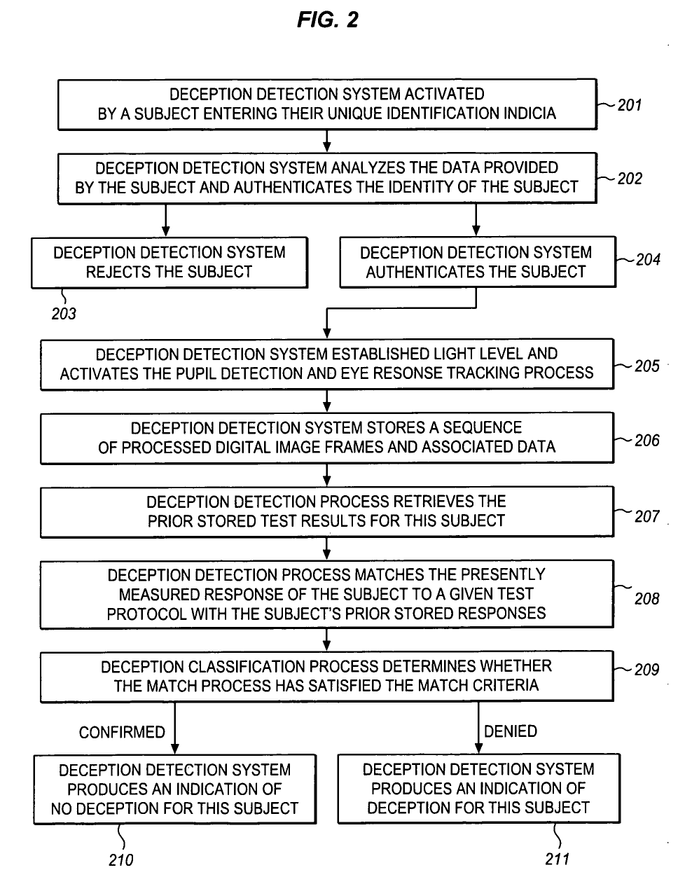

Deception Determination Process 106 can be used, for example, to take the pupil area and responsiveness output data from the present Pupil Detection and Response Process 105 to determine deception of the subject. FIG. 2 illustrates, in flow diagram form, the operation of the Deception Detection System 100. The Deception Detection System 100 is activated at step 201 by a subject entering their unique identification indicia or an examiner entering subject identification via a keypad, scanner device, wireless interface, etc., to distinguish this subject from others. The authentication of the subject can include a subject specific password and/or the scanning of an immutable physical characteristic of the subject, such as a fingerprint, iris scan, voiceprint, etc. The Deception Detection System 100 at step 202 analyzes the data provided by the subject and authenticates the identity of the subject based on previously stored identification criteria that have been stored for this subject. If there is a failure to match the subject with the stored identification criteria, the Deception Detection System 100 rejects the subject at step 203 and generates an alarm indication that the subject is not a valid user. A match results in the Deception Detection System 100 setting the ambient light level to controllably set a baseline size of the subject's pupil at step 204. In particular, the pupil size is biased by the data processor controlling the operation of the illumination devices 121, 122 to set the subject's pupil size at a point where changes in pupil size can most reliably be detected. Once this process is completed, the Deception Detection System 100 proceeds at step 205 to activate the Pupil Detection and Response Process 105. The collection of this data is a condition precedent to the operation of the Deception Determination Process 106, which is described below.

### Operation of the Pupil Detection and Response Process

The Pupil Detection and Response Process 105 processes each digital image frame in a sequence of digital image frames received from the video capture device and outputs data indicative of the pupil area, such as the digital image frame with the pupil boundary highlighted and with crosshairs delineating the pupil centroid location. The video capture device generates a time sequence of digital image frames, each of which is an image of at least one of the subject's eyes. The video capture device can create a single image of one eye, or both eyes, or paired image frames of the eyes, and the Pupil Detection and Response Process 105 may track the pupil in one or both of the subject's eyes. The present description refers to the processing of an image of one of the subject's eyes as the common descriptive example that is used for simplicity's sake and is intended to include all possible combinations of eye imaging and frame generation in all of these possible scenarios.

### Pupil Bias System

The Pupil Bias System 113 operates in cooperation with the Pupil Detection and Response Process 105 to establish a baseline illumination for the operation of the Deception Detection System 100. As noted above, the Pupil Bias System 113 provides the ability to bias the subject's pupil size by providing a controllable level of illumination to ensure that the subject's pupils are not overly or under dilated, which would limit the ability of the Pupil Detection and Response Process 105 to detect the greatest range of pupil reaction. The present Deception Detection System 100 provides a small amount of light that slightly constricts the subject's pupil but does not compromise its ability to be physiologically dilated. The Pupil Bias System 113 can either use the illumination devices 123, 124 in the self-contained headset 101 which eliminates ambient light and provides the pupil biasing illumination all within the same ‘chamber’. Alternatively, the Pupil Bias System 113 can control the ambient light in the room where the subject is being questioned to a low-light level that would cause the pupil to slightly constrict, but not enough to preclude its dilation. This control of ambient lighting can either be implemented via interconnection with the existing lighting control circuitry in the room or by providing feedback to the examiner to enable the examiner to manually control the ambient lighting.

### Deception Determination Process

Upon completion of the image data processing by Pupil Detection and Response Process 105, Deception Detection System 100 can optionally store a sequence of processed digital image frames, each of which contains highlighted pixels corresponding to the extracted pupil region, and cross-hairs drawn at the coordinates of the pupil's centroid location, in a memory. The data indicative of pupil area and XY-centroid location can also optionally be stored for use in the determination of deception. The Deception Determination Process 106 uses this pupil area and location data to determine whether a subject is attempting to provide false answers to the questions posed by the examiner.

The Deception Detection System 100 typically stores a set of questions, either custom generated for this subject or a brief series of pre-recorded test questions, in a question database 103 for use by the examiner. Deception Determination Process 106 maintains one or more test responses, indicative of a normal eye response or the subject's normal eye response to a set of baseline questions. At step 207, the Deception Determination Process 106 retrieves the normal test results. By matching the presently measured response of the subject to a given question in the set of questions at step 208 with the normal responses, the Deception Determination Process 106 can make an accurate determination of the subject's deception. The matching can be based on an algorithmic computation of the subject's pupil size in each digital image frame of the successive digital image frames and whether the pupil size during the pendency of the present question is within a predetermined range of response. Thus, there is an interaction between the examiner 2 and the Deception Detection System 100 in that the examiner 2 must provide an indication that the presentation of the question has begun, ended and also the start and conclusion of the subject's response to each question. This enables the data correlation process 107 to match the digital image frames to each event in the above-noted sequence of events associated with each question. The pupil size response to a presented question and the subject's answer to that question provide a precise determination of deception. Thus, at step 209, the Deception Determination Process 106 determines whether the match process executed at step 208 has satisfied the match criteria and, if a match is confirmed, at step 210 the Deception Detection System 100 produces an indication of no deception for this subject. However, if the match process executed at step 208 fails to satisfy the match criteria, the Deception Detection System 100 produces an indication of deception for this subject at step 211.

The Deception Detection System 100 could be a self-contained booth that presents a brief series of pre-recorded test questions records, records an optimal subset of electrodermal, oculomotor, cardiovascular, respiration, and/or thermal responses, and indicates if the responses to questions of interest were significant and this subject merits further investigation. The Deception Detection System 100 can present pre-recorded instructions and test questions to the subject and collect and analyze the physiological data. The Deception Detection System 100 can then use data correlation process 107 to create a combined test result and reports the relative strength of the subject's psychophysiological responses to the questions and classifies the subject immediately after data collection is complete. Physiological measures that require little or no contact with the subject or operator intervention (e.g., pupil diameter) are preferred over measures that require attachment of multiple sensors to the skin by an operator (e.g., skin conductance).

Since a subject's pupil diameter is correlated with increases in skin conductance, pupil diameter may be as useful as skin conductance for detecting deception. The advantage of measuring a subject's pupil diameter is that it may be measured remotely with off-the-shelf eye-trackers and, unlike skin conductance, does not require application of wet surface electrodes. In addition, the Deception Detection System 100 incorporates algorithms that detect the relatively large, high frequency and high amplitude changes in physiological signals that characterize movement artifacts and monitors respiration for deep breaths. Depending on the size and frequency of such artifacts, the system automatically reminds the subject to remain still or breathe normally during the test. The system automatically ignores measurements of potentially contaminated physiological signals. The system also adjusts the question sequences automatically during data collection to ensure that the decision model has a sufficient number of artifact-free responses on which to base its decision.

### SUMMARY

The present Deception Detection System accurately and automatically tracks the size, location, and responsiveness of a subject's pupil(s) in response to a set of questions posed to the subject by an examiner.

What is claimed as new and desired to be protected by Letters Patent of the United States is:

1. A Deception Detection System for automatically identifying a subject's attempt at deception in response to being presented with a set of questions by an examiner, comprising:
image means for generating a time-wise sequence of digital images of at least one of the subject's eyes, said sequence of digital images being of duration at least sufficient to include the time when said subject is being presented with a question by an examiner and when said subject is answering said question;
digital image processing means for computing a size of a pupil of at least one of said subject's eyes in said plurality of said digital images in said sequence of digital images; and
deception determining means, responsive to said determined size of said pupil in said plurality of said digital images in said sequence of digital images, for determining whether said subject is attempting to provide a deceptive answer to said question.

2. The Deception Detection System of claim 1 wherein said deception determining means comprises:
pupil responsiveness means for determining a dynamic response of said pupil size during said sequence of digital images.

3. The Deception Detection System of claim 2 wherein said deception determining means further comprises:
pattern means for comparing said dynamic response of said pupil size during said sequence of digital images with a normal response of pupil size to identify an anomalous response of said subject's pupil.

4. The Deception Detection System of claim 2, further comprising:
display means for presenting said examiner with a visual representation of said dynamic response of said pupil size during said sequence of digital images.

5. The Deception Detection System of claim 1 wherein said deception determining means comprises:
pattern means for identifying a departure of said determined size of said pupil in said sequence of digital images against normal pupil size.

6. The Deception Detection System of claim 1, further comprising:
polygraph means for generating data indicative of at least one of Respiratory Rate, Blood Pressure/Heart Rate, and Galvanic Skin Resistance measurements of said subject, said data being at least sufficient to include the time when said subject is being presented with a question by an examiner and when said subject is answering said question; and
correlation means for correlating said data indicative of at least one of Respiratory Rate, Blood Pressure/Heart Rate, and Galvanic Skin Resistance measurements of said subject with said determined size of said pupil in said plurality of said digital images in said sequence of digital images.

7. The Deception Detection System of claim 6 wherein said deception determining means comprises:
pattern means for comparing said data indicative of at least one of Respiratory Rate, Blood Pressure/Heart Rate, and Galvanic Skin Resistance measurements of said subject and said determined size of said pupil in said plurality of said digital images in said sequence of digital images with a normal response of a subject to identify an anomalous response of said subject.

8. The Deception Detection System of claim 1, further comprising:
illumination control means for generating a level of illumination for at least one of said subject's eyes to bias said pupil to a predetermined size prior to administration of said questions.

9. A method for automatically identifying a subject's attempt at deception in response to being presented with a set of questions by an examiner, comprising:
generating a time-wise sequence of digital images of at least one of the subject's eyes, said sequence of digital images being of duration at least sufficient to include the time when said subject is being presented with a question by an examiner and when said subject is answering said question;
computing a size of a pupil of at least one of said subject's eyes in said plurality of said digital images in said sequence of digital images; and
determining, in response to said determined size of said pupil in said plurality of said digital images in said sequence of digital images, whether said subject is attempting to provide a deceptive answer to said question.

10. The method for automatically identifying a subject's attempt at deception of claim 9 wherein said step of determining deception comprises:
determining a dynamic response of said pupil size during said sequence of digital images.

11. The method for automatically identifying a subject's attempt at deception of claim 10 wherein said step of determining deception further comprises:
comparing said dynamic response of said pupil size during said sequence of digital images with a normal response of pupil size to identify an anomalous response of said subject's pupil.

12. The method for automatically identifying a subject's attempt at deception of claim 10, further comprising:
presenting a visual representation to said examiner of said dynamic response of said pupil size during said sequence of digital images.

13. The method for automatically identifying a subject's attempt at deception of claim 9 wherein said step of determining deception comprises:
identifying a departure of said determined size of said pupil in said sequence of digital images against normal pupil size.

14. The method for automatically identifying a subject's attempt at deception of claim 9, further comprising:
generating data indicative of at least one of Respiratory Rate, Blood Pressure/Heart Rate, and Galvanic Skin Resistance measurements of said subject, said data being at least sufficient to include the time when said subject is being presented with a question by an examiner and when said subject is answering said question; and
correlating said data indicative of at least one of Respiratory Rate, Blood Pressure/Heart Rate, and Galvanic Skin Resistance measurements of said subject with said determined size of said pupil in said plurality of said digital images in said sequence of digital images.

15. The method for automatically identifying a subject's attempt at deception of claim 14 wherein said step of determining deception comprises:
comparing said data indicative of at least one of Respiratory Rate, Blood Pressure/Heart Rate, and Galvanic Skin Resistance measurements of said subject and said determined size of said pupil in said plurality of said digital images in said sequence of digital images with a normal response of a subject to identify an anomalous response of said subject.

16. The method for automatically identifying a subject's attempt at deception of claim 9, further comprising:
generating a level of illumination for at least one of said subject's eyes to bias said pupil to a predetermined size prior to administration of said questions.
